(19) United States
(12) Patent Application Publication
(10) Pub. No.: US 2025/0362223 A1
(43) Pub. Date: Nov. 27, 2025

(54) OPTICAL RESONANT CAVITY AND GAS ABSORPTION SPECTRUM DETECTION DEVICE

(71) Applicant: JIANGSU XUHAI OPTO-ELECTRONIC TECHNOLOGIES CO., LTD., Jiangsu (CN)

(72) Inventors: Bo CHEN, Jiangsu (CN); Huijie XU, Jiangsu (CN); Junhua WEN, Jiangsu (CN); Zhiquan YANG, Jiangsu (CN)

(73) Assignee: JIANGSU XUHAI OPTO-ELECTRONIC TECHNOLOGIES CO., LTD., Jiangsu (CN)

(21) Appl. No.: 18/873,713

(22) PCT Filed: Sep. 20, 2022
(86) PCT No.: PCT/CN2022/119994

§ 371 Date: Dec. 10, 2024

(51) Int. Cl. G01N 21/31; G01N 33/00

(52) U.S. Cl. CPC G01N 21/31; G01N 33/0027; G01N 2201/0636; G01N 2201/08

(57) ABSTRACT

An optical resonant cavity and a gas absorption spectrum detection device. The optical resonant cavity includes: a first cavity mirror and a second cavity mirror. The first cavity mirror includes multiple reflection points, and at least one of all the reflection points of the first cavity mirror is an input reflection point. A reflective surface of the second cavity mirror is arranged opposite to a reflective surface of the first cavity mirror, and the second cavity mirror and the first cavity mirror forms the optical resonant cavity. The second cavity mirror includes multiple reflection points, and at least one of the reflection points of the first cavity mirror or second cavity mirror is an output reflection point.

### CROSS-REFERENCE TO THE RELATED APPLICATIONS

This application is the national phase entry of International Application No. PCT/CN2022/119994, filed on Sep. 20, 2022, the content of which is incorporated herein by reference in its entirety.

### TECHNICAL FIELD

The present application relates to the field of cavity enhance absorption spectroscopy (Cavity Enhance Absorption Spectroscopy, CEAS) technology, more particularly to an optical resonant cavity and a gas absorption spectrum detection device.

### BACKGROUND

Environmental protection, safety, industry and other fields have put forward higher requirements on the lower limit of gas absorption spectrum detection. To meet this requirement, a method of increasing the optical path is adopted in the gas absorption spectrum detection technology to improve the gas absorption rate and reduce the detection lower limit. However, under a limited volume, the optical path cannot be increased infinitely. CEAS technologies developed in recent years, such as the cavity ring-down spectroscopy (Cavity Ring-Down Spectroscopy, CRDS) technology, the incoherent broad band (Incoherent Broad Band, IBB) cavity enhance absorption spectroscopy (IBBCEAS) technology, the off axis integrating cavity output spectroscopy (Off Axis Integrating Cavity Output Spectroscopy, OA-ICOS) technology, etc., use the characteristics of continuous reflection of light in the optical resonant cavity to increase the effective optical path by 10²-10⁴ times under a limited volume, thereby the sensitivity of the gas absorption spectrum detection device is greatly improved.

However, the low light energy received by the photodetector is a common problem in the gas absorption spectrum detection device based on CEAS technology, which limits the improvement of the signal-to-noise ratio and sensitivity of the gas absorption spectrum detection device.

### SUMMARY

One of objectives of the embodiments of the present application is to provide an optical resonant cavity and a gas absorption spectrum detection device, which aims at solving the problem that the light energy received by the photodetector of the existing gas absorption spectrum detection device based on CEAS technology is relatively low, which limits the improvement of the signal-to-noise ratio and sensitivity of the gas absorption spectrum detection device.

To solve the above technical problem, technical solutions adopted by the embodiments of the present application are as follows:

In accordance with a first aspect of the embodiments of the present application, an optical resonant cavity is provided which includes:
a first cavity mirror, the first cavity mirror includes multiple reflection points, and at least one of all the reflection points of the first cavity mirror is an input reflection point; and
a second cavity mirror, a reflection surface of the second cavity mirror is arranged opposite to a reflection surface of the first cavity mirror, the second cavity mirror includes multiple reflection points, and at least one of all the reflection points of the first cavity mirror or the second cavity mirror is an output reflection point;
where a light beam is transmitted into the optical resonant cavity through the input reflection point, and after the light bean is reflected N times between the reflection point of the first cavity mirror and the reflection point of the second cavity mirror, a re-incident condition is satisfied and then a next reflection cycle is entered, and such cycle is repeated until an energy of the light beam in the optical resonant cavity is attenuated to 0, N≥4, and the re-incident condition is that the reflection position and reflection angle of the light beam in the optical resonant cavity are the same as the transmission position and transmission angle of the light beam when it is first transmitted into the optical resonant cavity; and
at least one of all the input reflection points and all the output reflection points is a target reflection point, and a transmittance of the target reflection point is greater than or equal to T, and transmittances of the remaining reflection points are equal to T₀, where T>T₀>0.

In one embodiment, at least one of all the input reflection points has a transmittance being greater than or equal to T, and the second cavity mirror comprises N/2 output reflection points.

In one embodiment, T=mT₀, m=(N−1)/2, and m>1.

In one embodiment, one of all the input reflection points and one of all the output reflection points respectively have a transmittance being greater than or equal to T.

In one embodiment, T=mT₀, m=N−2, and m>1.

In one embodiment, one of all the input reflection points or one of all the output reflection points has a transmittance being greater than or equal to T.

In one embodiment, T=mT₀, m=(N−1)/2, and m>1.

In one embodiment, at least one of all the input reflection points has a transmittance being greater than or equal to Tᵢₙ, and at least one of all the output reflection points has a transmittance being greater than or equal to Tₒᵤₜ, where Tᵢₙ≠Tₒᵤₜ, Tᵢₙ≥T, and Tₒᵤₜ≥T.

In one embodiment, for a target cavity mirror from the first cavity mirror and the second cavity mirror, reflection points with different transmittances are formed on the target cavity mirror based on an integrated coating method or a split coating method, and the target cavity mirror includes multiple reflection points with different transmittances.

In one embodiment, a method for forming the multiple reflection points with different transmittances on the target cavity mirror based on the integrated coating method is that: different film layers in different areas of the target cavity mirror are generated by using a mask in an integrated coating process; and
a method for forming the multiple reflection points with different transmittances on the target cavity mirror based on the split coating method is that: different areas of the target cavity mirror are separated into independent components, and different components are coated separately in a split coating process.

In one embodiment, the optical resonant cavity also includes:
at least one folding reflector, each of which has a reflective surface opposite to the reflective surface of the first cavity mirror or the reflective surface of the second cavity mirror, and the folding reflector includes multiple reflection points; and
after the light beam is transmitted into the optical resonant cavity through the input reflection point, and is reflected M times between the reflection points of the first cavity mirror, the reflection points of the folding reflector and the reflection points of the second cavity mirror, the re-incident condition is satisfied and the next reflection cycle is entered, and such cycle is repeated until the energy of the light beam is attenuated to 0, where M>N.

In one embodiment, at least one of all the reflection points of the first cavity mirror or the second cavity mirror is an output reflection point, and the output reflection point is the target reflection point.

In one embodiment, at least one of the first cavity mirror and the second cavity mirror is a concave reflector.

In accordance with a second aspect of the embodiments of the present application, a gas absorption spectrum detection device is provided, including:
the optical resonant cavity provided by the first aspect of the embodiment of the present application; and
a photodetector, the photodetector is configured to measure a light intensity of the light beam transmitted through the output reflection point, to obtain the absorption spectrum information of a gas in the optical resonant cavity according to the light intensity or a ring-down time of the light intensity.

In one embodiment, the gas absorption spectrum detection device also includes a converging lens. The light beam, after being transmitted to the converging lens through the output reflection point, is converged to the photodetector through the converging lens.

In one embodiment, the gas absorption spectrum detection device also includes a converging lens and a receiving optical fiber. The light beam, after being transmitted to the converging lens through the output reflection point, is converged to the receiving optical fiber through the converging lens and transmitted to the photodetector.

In one embodiment, the gas absorption spectrum detection device is implemented based on a cavity ring-down spectroscopy technology, an incoherent broadband cavity enhanced absorption spectroscopy technology or an off-axis integral cavity output spectroscopy technology.

The optical resonant cavity provided according to the first aspect of the embodiments of the present application includes a first cavity mirror and a second cavity mirror. The first cavity mirror includes multiple reflection points, at least one of all the reflection points of the first cavity mirror is an input reflection point, and a reflection surface of the second cavity mirror is arranged opposite to a reflection surface of the first cavity mirror. The second cavity mirror and the first cavity mirror constitute the optical resonant cavity, the second cavity mirror includes multiple reflection points, at least one of all the reflection points of the first cavity mirror or the second cavity mirror is an output reflection point. A light beam is transmitted into the optical resonant cavity through the input reflection point, and after being reflected at least 4 times between the reflection points of the first cavity mirror and the reflection points of the second cavity mirror, a re-incident condition is satisfied and then a next reflection cycle is entered, and such cycle is repeated until the energy of the light beam in the optical resonant cavity is attenuated to 0. The re-incidence condition is that: a reflection position and a reflection angle of the light beam in the optical resonant cavity are the same as a transmission position and a transmission angle of the light beam when the light beam is first transmitted into the optical resonant cavity. The transmittance of at least one of all the input reflection points and all the output reflection points is arranged to be greater than the transmittances of the remaining reflection points, so that the light energy output by the output reflection point can be enhanced, thereby the light energy coupled to the photodetector by the optical resonant cavity when the optical resonant cavity is applied to the gas detection device can be increased, which then can effectively improve the signal-to-noise ratio and sensitivity of the gas detection device.

It can be understood that the beneficial effects of the above-mentioned second aspect can be referred to the relevant description in the above-mentioned first aspect, which will not be repeated here.

### BRIEF DESCRIPTION OF THE DRAWINGS

To illustrate the technical solutions in the embodiments of the present application more clearly, the drawings required to be used in the description of the embodiments will be briefly introduced below. Obviously, the drawings in the following description are only some embodiments of the present application. For ordinary technicians in this field, other drawings may be obtained based on these drawings without exerting creative efforts.

FIG. 1 is a schematic diagram of a first structure of an optical resonant cavity and a gas absorption spectrum detection device provided in an embodiment of the present application;

FIG. 2 is a schematic diagram of a second structure of the optical resonant cavity and the gas absorption spectrum detection device provided in an embodiment of the present application; and

FIG. 3 is a schematic diagram of a third structure of the optical resonant cavity and the gas absorption spectrum detection device provided in an embodiment of the present application.

### DETAILED DESCRIPTION OF THE EMBODIMENTS

To enable persons skilled in the art to better understand the present application, the technical solutions in the embodiments of the present application will be clearly described in combination with the drawings in the embodiment of the present application. Obviously, the described embodiments are merely some embodiments, not all of the embodiments of the present application. Based on the embodiments in the present application, other embodiments obtained by ordinary technicians in the field without exerting creative efforts should all fall within the protection scope of the present application.

The term “including/comprising” and any variation thereof in the specification and claims of the present application and the above-mentioned drawings are intended to cover non-exclusive inclusions. In addition, the terms “first” and “second” are used to distinguish different objects rather than to describe a specific order.

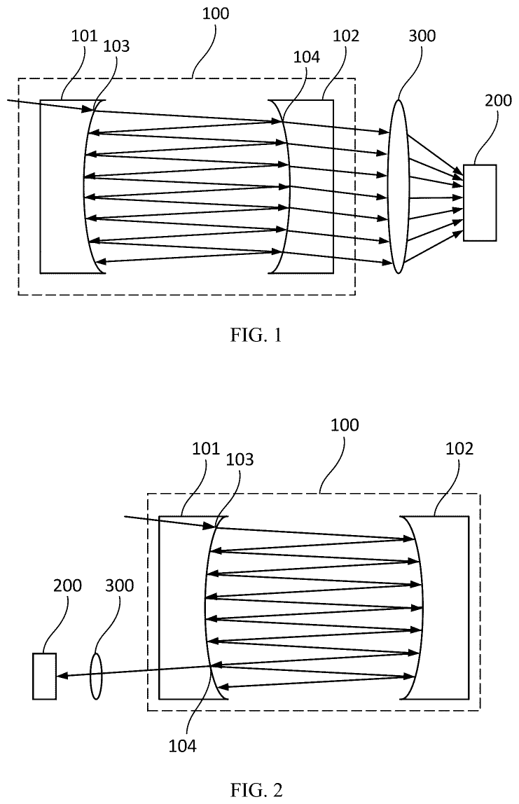

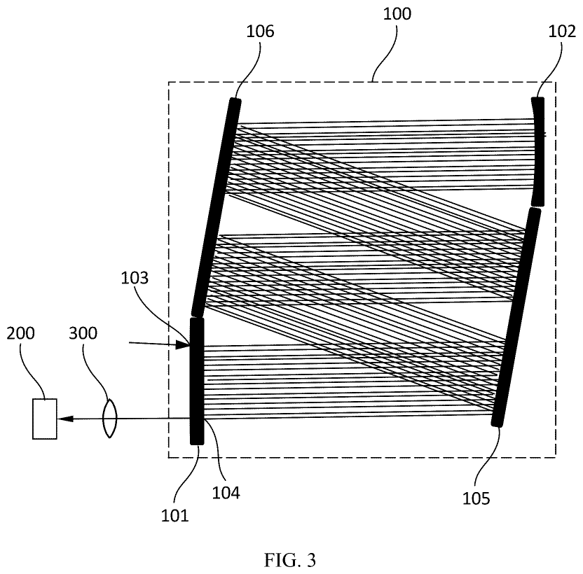

As shown in FIG. 1, FIG. 2 or FIG. 3, an embodiment of the present application provides an optical resonant cavity 100, which includes: a first cavity mirror 101 and a second cavity mirror 102.

The first cavity mirror 101 includes multiple reflection points, at least one of all the reflection points of the first cavity mirror 101 is an input reflection point 103.

A reflection surface of the second cavity mirror 102 is arranged opposite to a reflection surface of the first cavity mirror 101, the second cavity mirror 102 includes multiple reflection points, at least one of all the reflection points of the first cavity mirror 101 or the second cavity mirror 102 is an output reflection point 104.

In this embodiment, a light beam is transmitted into the optical resonant cavity 100 through the input reflection point 103, and after being reflected N times between the reflection points of the first cavity mirror 101 and the reflection points of the second cavity mirror 102, a re-incident condition is satisfied and then a next reflection cycle is entered, this cycle is repeated until the energy of the light beam in the optical resonant cavity is attenuated to 0, where N≥4, and the re-incident condition is: a reflection position and a reflection angle of the light beam in the optical resonant cavity 101 are the same as a transmission position (i.e., a position of the input reflection point 103) and a transmission angle θ of the light beam when the light beam is first transmitted into the optical resonant cavity 100.

The transmittance of at least one of all the input reflection points 103 and all the output reflection points 104 is greater than or equal to T, and the transmittances of the remaining reflection points are smaller than or equal to T₀, T>T₀>0.

In applications, the reflection point is a location point on the cavity mirror for reflecting the light beam. The input reflection point is a location point on the cavity mirror for inputting of the light beam from the light source and reflecting the light beam. The output reflection point is a location point on the cavity mirror for reflecting the light beam and outputting the light beam to the photodetector. The remaining reflection points except the input reflection point and the output reflection point are defined as ordinary reflection points. Reflection points whose transmittances are greater than or equal to T among all the input reflection points and all the output reflection points are defined as target reflection points, and the remaining reflection points whose transmittances are equal to T₀ include input reflection points, output reflection points and ordinary reflection points except the target reflection points.

In applications, the position and number of the input reflection point and the output reflection point of the optical resonant cavity may be set according to actual needs, which is specifically related to the type of the optical resonant cavity. As long as the input reflection point is arranged on the first cavity mirror, the output reflection point may be arranged on the first cavity mirror or the second cavity mirror. The input reflection point and the output reflection point may both be arranged on the first cavity mirror, in this case, the second cavity mirror is only provided with the ordinary reflection points. The input reflection point and the output reflection point may be the same reflection point (defined as an input-output reflection point), and at least one of all the reflection points of the first cavity mirror may be the input-output reflection point for inputting and outputting of the light beam.

In applications, when the number of target reflection points is at least two, the transmittances of these target reflection points may be the same or different. Due to the limitations of modern coating technology, the maximum reflectivity R of the cavity mirror can usually reach 0.99999, and then the difficulty and cost of increasing the reflectance are sharply increased, the corresponding T₀ (i.e. 1-R) can be as low as 0.00001, that is, the minimum T₀ can reach a magnitude of 10⁻⁵. T can reach a magnitude of 10⁻⁵ to 10⁻³, for example, can be between 0.0009-0.005.

In applications, the transmittances of all target reflection points are greater than or equal to T, which may specifically include but is not limited to the following situations:

In a first situation, the transmittances of all target reflection points are equal, for example, the transmittances of all target reflection points are equal to T.

In a second situation, the transmittances of all target reflection points are unequal or partially equal. For example, the transmittances of the target reflection points in all the input reflection points are equal to Tᵢₙ, and the transmittances of the target reflection points in all the output reflection points are equal to Tₒᵤₜ. Or alternatively, the transmittances of the target reflection points in all the input reflection points are greater than or equal to Tᵢₙ and are unequal or partially equal, and the transmittances of the target reflection points in all the output reflection points are greater than or equal to Tₒᵤₜ and are unequal or partially equal, where Tᵢₙ≠Tₒᵤₜ, Tᵢₙ≥T, and Tₒᵤₜ≥T.

In applications, the light beam is transmitted into the optical resonant cavity through the input reflection point, and after being reflected N times between the reflection point of the first cavity mirror and the reflection point of the second cavity mirror, the re-incident condition is satisfied, and then a next reflection cycle is entered where the same reflection path is used to reflect the light beam again between the reflection points of the first cavity mirror and the reflection points of the second cavity mirror, and the cycle is repeated until the energy of the light beam is attenuated to 0.

In applications, the target reflection point is provided having a transmittance being greater than that of the remaining reflection points, so that an average reflectance of all reflection points will be slightly lowered, and then an effective optical path will be slightly lowered, which has a negative impact on the signal-to-noise ratio. However, this technical means also greatly increases the light energy output by the output reflection point, thereby the light energy coupled to the photodetector by the optical resonant cavity when the optical resonant cavity is applied to the gas absorption spectrum detection device is improved, which has a positive impact on improving the signal-to-noise ratio of the gas absorption spectrum detection device. Since the positive impact is far greater than the negative impact, the signal-to-noise ratio will also be greatly improved. The larger the number N of single-cycle reflection points, the larger the improvement multiple of the signal-to-noise ratio.

In one embodiment, at least one of all the input reflection points has a transmittance being greater than or equal to T, and the second cavity mirror includes N/2 output reflection points. The relationship between T and T₀ may be that: T=m T₀, m=(N−1)/2, m>1.

In applications, at least one target reflection point for inputting of the light beam may be provided only in the first cavity mirror, and N/2 (i.e., at least two) output reflection points for outputting the light beam may be provided on the second cavity mirror, and the transmittance of the target reflection point may be greater than or equal to (N−1)/2 (i.e., at least 1.5) times T₀.

In one embodiment, one of all the input reflection points and one of all the output reflection points respectively have a transmittance being greater than or equal to T. The relationship between T and T₀ may be that: T=m T₀, m=N−2, m>1.

In applications, only one target reflection point for inputting of the light beam may be provided on the first cavity mirror, and in the meantime, one target reflection point for outputting the light beam may be provided on the first cavity mirror or the second cavity mirror. The target reflection point for inputting of the light beam and the target reflection point for outputting the light beam may be the same target reflection point when the target reflection point for inputting of the light beam and the target reflection point for outputting the light beam are both provided on the first cavity mirror. The transmittance of the target reflection point may be greater than or equal to (N−2) (i.e., at least 2) times T₀.

In one embodiment, one of all the input reflection points or one of all the output reflection points has a transmittance being greater than or equal to T. The relationship between T and T₀ may be that: T=m T₀, m=(N−1)/2, m>1.

In applications, only one target reflection point for inputting of the light beam may be provided on the first cavity mirror, or only one target reflection point for outputting the light beam may be provided on the first cavity mirror or the second cavity mirror. The transmittance of the target reflection point may be greater than or equal to (N−1)/2 (that is, at least 1.5) times T₀.

In one embodiment, for a target cavity mirror including reflection points with different transmittances of the first cavity mirror or the second cavity mirror, reflection points with different transmittances are formed on the target cavity mirror based on an integrated coating method or a split coating method.

In applications, for each of the first cavity mirror and the second cavity mirror, if the cavity mirror includes reflection points with different transmittances, the reflection points with different transmittances may be formed on the cavity mirror based on the integrated coating method or the split coating method. If the cavity mirror only includes reflection points with the same transmittance, the reflection points with the same transmittance may be formed on the cavity mirror based on the integrated coating method. The cavity mirror including reflection points with different transmittances is defined as the target cavity mirror.

In one embodiment, based on the integrated coating method, the reflection points with different transmittances are formed on the target cavity mirror by: using a mask, in an integrated coating process, to generate different film layers in different areas of the target cavity mirror

Based on the split coating method, the reflection points with different transmittances are formed on the target cavity mirror by: separating different areas of the target cavity mirror into independent components and coating the different components separately in a split coating process.

In applications, different areas of the target cavity mirror are the location points where reflection points with different transmittances are located. The number of independent components separated from different areas of the target cavity mirror is determined by the number of the target reflection points and the remaining reflection points included by the target cavity mirror. The number of independent components ranges from the number of target reflection points plus 1 to the number of target reflection points plus the number of remaining reflection points. That is, the location point where each target reflection point is located is separated into one area, and all remaining reflection points are separated into one area or each remaining reflection point is separated into one area. In one embodiment, the first cavity mirror is a concave reflector, and the second cavity mirror is a concave reflector or a plane reflector.

In applications, the number of the input reflection point and the output reflection point and their locations, as well as the type of the first cavity mirror and the second cavity mirror may be adjusted according to actual needs.

FIG. 1 shows a structural schematic diagram of a first optical resonant cavity based on a Herriott Cell.

Herein, the first cavity mirror 101 is a concave reflector, and one of all the reflection points of the first cavity mirror 101 is an input reflection point 103.

The second cavity mirror 102 is a concave reflector, and multiple reflection points among all the reflection points of the second cavity mirror 102 are output reflection points 104, and the first cavity mirror 101 and the second cavity mirror 102 constitute the Herriott Cell.

In applications, the number of reflections of the light beam in the optical resonant cavity shown in FIG. 1 may usually reach 50-100 times, and the first cavity mirror and the second cavity mirror are a pair of high-reflectivity concave reflectors, and the reflectivity may reach more than 99%.

FIG. 2 exemplarily shows a structural schematic diagram of a second optical resonant cavity based on the Herriott cell.

Herein, the first cavity mirror 101 is a concave reflector, one of all the reflection points of the first cavity mirror 101 is an input reflection point 103, and another one of the reflection points of the first cavity mirror 101 is an output reflection point 104.

The second cavity mirror 102 is a concave reflector, and the first cavity mirror 101 and the second cavity mirror 102 constitute the Herriott cell.

As shown in FIG. 3, in one embodiment, the optical resonant cavity 100 also includes at least one folding reflector.

The reflection surface of each folding reflector is arranged opposite to the reflection surface of the first cavity mirror 101 or the reflection surface of the second cavity mirror 102, and the folding reflector includes multiple reflection points.

The light beam is transmitted into the optical resonant cavity 100 through the input reflection point 103, and after being reflected M times at the reflection point of the first cavity mirror 101, between the reflection points of the folding reflector, and at the reflection point of the second cavity mirror 102, the re-incident condition is satisfied and the next reflection cycle is entered, and the cycle is repeated until the energy of the light beam in the optical resonant cavity is attenuated to 0, where M>N.

In applications, at least one folding reflector may be additionally added on the basis of the first cavity mirror and the second cavity mirror to increase the number of reflections of the light beam in the optical resonant cavity. In case that that the straight-line distance between the first cavity mirror and the second cavity mirror remains unchanged, the total optical path of the light beam propagating in the optical resonant cavity can be increased by adding the folding reflector. In case that the total optical path remains unchanged, the volume of the optical resonant cavity can be reduced by adding the folding reflector, and the optical path volume ratio of the optical resonant cavity can be improved.

In one embodiment, for an optical resonant cavity including a folding reflector, at least one of all reflection points of the first cavity mirror or the second cavity mirror is an output reflection point, and the output reflection point is a target reflection point.

FIG. 3 exemplarily shows a structural schematic diagram of a third optical resonant cavity 100 including a first folding reflector and a second folding reflector.

Herein, the first cavity mirror 101 is a plane reflector, one of all reflection points of the first cavity mirror 101 is an input reflection point 103, and another one of the reflection points of the first cavity mirror 101 is an output reflection point 104.

The second cavity mirror 102 is a concave reflector.

The reflection surface of the first plane folding reflector 105 is arranged opposite to the reflection surface of the first cavity mirror 101.

The reflection surface of the second plane folding reflector 106 is arranged opposite to the reflection surface of the second cavity mirror 102 and is parallel to the reflection surface of the first plane folding reflector 105.

The optical resonant cavity provided by the embodiments of the present application has at least the following characteristics:

In a first aspect, when the light beam is an incoherent light beam, the absorption signal of the optical resonant cavity is greatly enhanced. Assuming that the number of reflections in a single reflection cycle is N, where N≥4, an average transmittance of the optical resonant cavity is T′, an average reflectance is R′, a cavity length is L, the transmittance of the input reflection point is Tᵢₙ, the reflectance of the input reflection point is Rᵢₙ, where Rᵢₙ=1−Tᵢₙ, the number of output reflection points is n, the transmittance of the output reflection point is Tₒᵤₜ, the reflectance of the output reflection point is Rₒᵤₜ, where Rₒᵤₜ=1−Tₒᵤₜ, the transmittances of other reflection points are T₀, the reflectances of the other reflection points are R₀, where R₀=1−T₀, Tᵢₙ>T₀ or Tₒᵤₜ>T₀, then an equivalent absorption optical path L_{eff} of the optical resonant cavity is expressed as:

$${L}_{eff} = L / ( 1 - R ’ ) = L / T ’ \qquad ( Formula 1 )$$

The average transmittance T′ is expressed as:

$$T ’ = [ ( N - n - 1 ) {T}_{0} + {T}_{i n} + n {T}_{out} ] / N \qquad ( Formula 2 )$$

During a continuous reflecting of the light beam between the cavity mirrors, a small amount of light is continuously transmitted out of the optical resonant cavity at each of the n output reflection points, and is converged by the converging lens to reach the photodetector. The energy efficiency η is expressed as:

$$η = n {T}_{i n} {T}_{out} / [ ( N - n - 1 ) {T}_{0} + {T}_{i n} + n {T}_{out} ] \qquad ( Formula 3 )$$

By substituting Formula 1 into Formula 3, the relative signal-to-noise ratio SNRr can be obtained as:

$$SNRr = {L}_{eff} {η}^{1 / 2} / L = {( {nT}_{i n} {T}_{out} )}^{1 / 2} / T ’ {[ ( N - n - 1 ) {T}_{0} + {T}_{i n} + n {T}_{out} ]}^{1 / 2} \qquad ( Formula 4 )$$

Furthermore, to maintain generality, let Tᵢₙ=m₁T₀, Tₒᵤₜ=m₂T₀, m₁≥1, m₂≥1, then the relative signal-to-noise ratio SNRr is expressed as:

$$SNRr = {N ( 2 n {m}_{1} {m}_{2} )}^{1 / 2} / {( N - n - 1 + {m}_{1} + n {m}_{2} )}^{3 / 2} {( 2 {T}_{0} )}^{1 / 2} = K / {( 2 {T}_{0} )}^{1 / 2} \qquad ( Formula 5 )$$

$$K = {N ( 2 n {m}_{1} {m}_{2} )}^{1 / 2} / {( N - n - 1 + {m}_{1} + n {m}_{2} )}^{3 / 2} \qquad ( Formula 6 )$$

It can be seen from Formula 6 that for the first optical resonant cavity shown in FIG. 1, when n=N/2, m₁=m₂=1, the relative signal-to-noise ratio of the first optical resonant cavity SNRr=1/(2T₀)^{1/2}, that is, K=1. Therefore, for a given N, by reasonably designing the values of n, m₁, and m₂, K>1 can be achieved, thereby improving the relative signal-to-noise ratio of the first optical resonant cavity. The value of K represents the improvement multiple of the relative signal-to-noise ratio. For example:

When n=N/2, m₁=m, m₂=1, m>1, that is, the transmittance of the input reflection point is greater than the transmittances of the remaining reflection points, then:

$$K = {{m}^{1 / 2} [ 1 + ( m - 1 ) / N ]}^{- 3 / 2} \qquad ( Formula 7 )$$

By taking the derivative of K in Formula 7 with respect to m, the extreme value Kₘₐₓ of K can be obtained as:

$${K}_{max} = 2 {N ( N / 3 )}^{1 / 2} / ( 3 N - 3 ) , m = ( N - 1 ) / 2 \qquad ( Formula 8 )$$

When n=1, m₁=m₂=m, m>1, that is, the transmittance of the input reflection point and the transmittance of the output reflection point are both greater than the transmittances of the remaining reflection points, then:

$$K = {( 2 / N )}^{1 / 2} {m [ 1 + ( 2 m - 2 ) / N ]}^{- 3 / 2} \qquad ( Formula 9 )$$

By taking the derivative of K in Formula 9 with respect to m, the extreme value Kₘₐₓ of K can be obtained as:

$${K}_{max} = {[ 2 / ( 3 N - 6 ) ]}^{1 / 2} N / 3 , m = N - 2. \qquad ( Formula 10 )$$

In a second aspect, when the light beam is a coherent light beam, assuming that the number of output reflection points is n, and n=1, the number of reflections in a single reflection cycle is N, and a relative coupling efficiency Tᵣ is the light energy efficiency ratio of the folded cavity (i.e., an optical resonant cavity including at least one folded reflector) coupled to the photodetector relative to the straight cavity (i.e., a reflective cavity formed by two plane reflectors arranged oppositely), and the following two cases are analyzed.

In a first case:

Assuming that the transmittances of the input reflection point and the output reflection point are T₁, and the transmittances of the remaining reflection points are T₀, T₁=mT₀, m>1.

Considering the free spectral range FSR, the change of fineness with N, and the change of the cavity mode peak coupling efficiency, the relative coupling efficiency Tᵣ may be expressed as:

$${T}_{r} = 4 {m}^{2} / [ N ( N + 2 m - 2 ) ] \qquad ( Formula 11 )$$

When m=1, N=2, the optical resonant cavity is a straight cavity, substituting these values into formula 11, then Tᵣ=1 can be obtained.

When m=1, N≥4, the optical resonant cavity is a folded cavity or an off-axis integrating cavity (for example, the first optical resonant cavity shown in FIG. 1 or the second optical resonant cavity shown in FIG. 2), substituting these values into formula 11, then Tᵣ=4/N² can be obtained.

The relative signal-to-noise ratio SNRr is expressed as:

$$SNRr = {L}_{eff} {{T}_{r}}^{1 / 2} / L = 2 K / ( {NT}_{0} ) \qquad ( Formula 12 )$$

$$K = {m [ 1 + ( 2 m - 2 ) / N ]}^{- 3 / 2} \qquad ( Formula 13 )$$

When m=1, the optical resonant cavity is a straight cavity or a folded cavity, then K=1, SNRr=2/(NT₀).

When N≥4, m>1, K>1, taking the derivative of K in Formula 13 with respect to m, then the extreme value Kₘₐₓ of K can be obtained as:

$${K}_{max} = {[ N / ( 3 N - 6 ) ]}^{1 / 2} N / 3 , m = N - 2. \qquad ( Formula 14 )$$

In a second case:

Assuming that one of the input reflection points or the output reflection points has a transmittance of T₁, that is, there is only one target reflection point, and the transmittances of the remaining reflection point are T₀, T₁=mT₀, m>1.

Considering the free spectral range FSR, the change of fineness with N, and the change of cavity mode peak coupling efficiency, the relative coupling efficiency Tᵣ may be expressed as:

$${T}_{r} = 4 m / [ N ( N + m - 1 ) ] \qquad ( Formula 15 )$$

When m=1, N=2, the optical resonant cavity is a straight cavity, substituting these values into Formula 18, Tᵣ=1 can be obtained.

When m=1, N≥4, the optical resonant cavity is a folded cavity or an off-axis integrating cavity (for example, the first optical resonant cavity shown in FIG. 1 or the second optical resonant cavity shown in FIG. 2), and substituting these values into formula 18, Tᵣ=4/N² can be obtained.

The relative signal-to-noise ratio SNRr is expressed as:

$$SNRr = {L}_{eff} {{T}_{r}}^{1 / 2} / L = 2 K / ( {NT}_{0} ) \qquad ( Formula 16 )$$

$$K = {{m}^{1 / 2} [ 1 + ( m - 1 ) / N ]}^{- 3 / 2} \qquad ( Formula 17 )$$

When m=1, the optical resonant cavity is a straight cavity or a folded cavity, then K=1, SNRr=2/(NT₀).

When N≥4, m>1, K>1, taking the derivative of K in formula 13 with respect to m, and the extreme value Kₘₐₓ of K can be obtained as:

$${K}_{max} = 2 {N ( N / 3 )}^{1 / 2} / ( 3 N - 3 ) , m = ( N - 1 ) / 2. \qquad ( Formula 18 )$$

In one embodiment, for the first optical resonant cavity as shown in FIG. 1, the light beam may be a coherent light beam or an incoherent light beam (for example, an incoherent broadband light beam). In the case where the light beam is a coherent light beam, since the number of output reflection points is multiple, the interference effect is greatly smoothed. Therefore, the coherent light beam can be approximated by the same method as the incoherent light beam.

When Tᵢₙ=mT₀, Tₒᵤₜ>T₀, m>1, it can be obtained according to formulas 6 and 7 that:

$$K = {{m}^{1 / 2} [ 1 + ( m - 1 ) / N ]}^{- 3 / 2} \qquad ( Formula 19 )$$

When N≥4, by reasonably designing the value of m, K>1 can be achieved.

When m=(N−1)/2, it can be determined according to formula 8 that the larger the value of N, the larger the value of Kₘₐₓ, for example:

$$If N = 50 , then {K}_{max} = 2.78 .$$

$$If N = 100 , then {K}_{max} = 3.89 .$$

As for the first optical resonant cavity shown in FIG. 1, when the value of Nis between 50-100, the relative signal-to-noise ratio is increased by 2.78-3.89 times. The larger the value of N, the larger the improvement multiple of the relative signal-to-noise ratio.

When T₀=0.01%, R₀=99.99%, N=50, it can be determined according to formula 19 that an optimal value of m is 24.5. At this time, Tᵢₙ=24.5×0.01%=0.245%, and Rᵢₙ=99.755%.

In one embodiment, for an optical resonant cavity in which one of all reflection points of the first cavity mirror or the second cavity mirror is an output reflection point (for example, the second optical resonant cavity shown in FIG. 2), the light beam may be a coherent light beam or an incoherent light beam (for example, an incoherent broadband light beam).

When Tᵢₙ=Tₒᵤₜ=mT₀, m>1, since the number of output reflection points is one, that is, n=1, then:

$$\begin{matrix}1 ) & \\ If the light beam is an incoherent light beam , \\ K = {( 2 / N )}^{1 / 2} {m [ 1 + ( 2 m - 2 ) / N ]}^{- 3 / 2} & ( Formula 9 )\end{matrix}$$

$$Correspondingly , {K}_{max} = {[ 2 / ( 3 N - 6 ) ]}^{1 / 2} N / 3 , m = N - 2 \qquad ( Formula 10 )$$

$$If N = 50 , then {K}_{max} = 1.96 .$$

$$If N = 100 , then {K}_{max} = 2.75 .$$

As for the second optical resonant cavity shown in FIG. 2, when the value of N is between 50 and 100, the relative signal-to-noise ratio is improved by a multiple of 1.96 to 2.75. The larger the value of N, the greater the improvement of the relative signal-to-noise ratio.

$$\begin{matrix}2 ) & \\ If the light beam is a coherent beam , \\ K = {m [ 1 + ( 2 m - 2 ) / N ]}^{- 3 / 2} & ( Formula 13 )\end{matrix}$$

$$Correspondingly , {K}_{max} = {[ N / ( 3 N - 6 ) ]}^{1 / 2} N / 3 , m = N - 2 \qquad ( Formula 14 )$$

$$If N = 50 , then {K}_{max} = 9.82 .$$

$$If N = 100 , then {K}_{max} = 19.4 .$$

As for the second optical resonant cavity shown in FIG. 2, when the value of N is between 50 and 100, the relative signal-to-noise ratio is improved by a multiple of 9.82 to 19.4. The larger the value of N, the greater the improvement of the relative signal-to-noise ratio.

3) If T₀=0.01%, R₀=99.99%, N=50, the optimal value of m is m=N−2=48. At this time, Tᵢₙ=Tₒᵤₜ=48×0.01%=0.48%, Rᵢₙ=Rₒᵤₜ=99.52%.

In one embodiment, for a folded cavity (e.g., the third optical resonant cavity shown in FIG. 3), the light beam may be a coherent light beam or an incoherent light beam (e.g., an incoherent broadband light beam).

When Tᵢₙ=Tₒᵤₜ=mT₀, m>1, since the number of output reflection points is one, that is, n=1, then:

$$\begin{matrix}1 ) & \\ If the light beam is an incoherent light beam , \\ K = {( 2 / N )}^{1 / 2} {m [ 1 + ( 2 m - 2 ) / N ]}^{- 3 / 2} & ( Formula 9 )\end{matrix}$$

$$Correspondingly , {K}_{max} = {[ 2 / ( 3 N - 6 ) ]}^{1 / 2} N / 3 , m = N - 2 \qquad ( Formula 10 )$$

$$If N = 50 , then {K}_{max} = 1.96 .$$

$$If N = 100 , then {K}_{max} = 2.75 .$$

As for the third optical resonant cavity shown in FIG. 3, when the value of N is between 50-100, the relative signal-to-noise ratio is improved by a multiple of 1.96-2.75.

The larger the value of N, the greater the improvement of the relative signal-to-noise ratio.

$$\begin{matrix}2 ) & \\ If the light beam is a coherent light beam , \\ K = {m [ 1 + ( 2 m - 2 ) / N ]}^{- 3 / 2} & ( Formula 13 )\end{matrix}$$

$$Correspondingly , {K}_{max} = {[ N / ( 3 N - 6 ) ]}^{1 / 2} N / 3 , m = N - 2 \qquad ( Formula 14 )$$

$$If N = 50 , then {K}_{max} = 9.82 .$$

$$If N = 100 , then {K}_{max} = 19.4 .$$

As for the third optical resonant cavity shown in FIG. 3, when the value of N is between 50 and 100, the relative signal-to-noise ratio is improved by a multiple of 9.82 to 19.4. The larger the value of N, the greater the improvement of the relative signal-to-noise ratio.

3) If T₀=0.001%, R₀=99.999%, and N=100, the optimal value of m is m=N−2=98. At this time, Tᵢₙ=Tₒᵤₜ=98×0.001%=0.098%, Rᵢₙ=Rₒᵤₜ=99.902%. It can be seen that the use of folded reflectors to form a folded cavity can further increase the value of N, thereby the improvement multiple of the relative signal-to-noise ratio K is further increased, and at the same time the free spectral range FSR can be reduced, and the resolution of the gas absorption spectrum detection device can be improved.

As shown in FIG. 1, FIG. 2 or FIG. 3, an embodiment of the present application also provides a gas absorption spectrum detection device which includes:
an optical resonant cavity 100; and
a photoelectric detector 200. The photoelectric detector 200 is configured to measure a light intensity of the light beam transmitted through the output reflection point 104, to obtain absorption spectrum information of a gas in the optical resonant cavity 100 according to the light intensity or the ring-down time of the light intensity.

In applications, by adopting the optical resonant cavity provided by the embodiment of the present application, the light intensity of the light beam coupled to the photoelectric detector can be increased, thereby the signal-to-noise ratio of the gas absorption spectrum detection device can be effectively improved, which further improves the accuracy of the absorption spectrum information of the acquired gas.

As shown in FIG. 1, FIG. 2 or FIG. 3, in one embodiment, the gas absorption spectrum detection device also includes a converging lens 300. The light beam, after being transmitted to the converging lens 300 through the output reflection point 104, is converged to the photoelectric detector 200 through the converging lens 300.

In one embodiment, the gas absorption spectrum detection device may also include a receiving optical fiber. The light beam, after being transmitted to the converging lens through the output reflection point, is converged to the receiving optical fiber through the converging lens and transmitted to the photoelectric detector.

In applications, the light beam outputted from the optical resonant cavity is converged by a converging lens and then coupled to a photodetector, or transmitted to the photodetector via a receiving optical fiber, which can reduce the area of the receiving surface of the photodetector, thereby the volume of the gas absorption spectrum detection device can be effectively reduced. This arrangement is particularly suitable for the case where the number of output reflection points is multiple.

In the gas absorption spectrum detection device shown in FIG. 2, as only a small-aperture converging lens provided at one output reflection point of the optical resonant cavity is in need, thus the volume of the gas absorption spectrum detection device is reduced.

In one embodiment, the gas absorption spectrum detection device is implemented based on cavity ring-down spectroscopy technology, incoherent broadband cavity enhanced absorption spectroscopy technology, or off-axis integral cavity output spectroscopy technology.

The above embodiments are merely some preferable embodiments of the present application which are not intended to limit the present application. Any modifications, equivalent substitutions, and improvements made within the spirit and principles of the present application shall all be included within the protection scope of the present application.

1. An optical resonant cavity, comprising:
a first cavity mirror, the first cavity mirror comprising multiple reflection points, the multiple reflection points of the first cavity mirror comprising at least one input reflection point; and
a second cavity mirror, a reflection surface of the second cavity mirror arranged opposite to a reflection surface of the first cavity mirror, the second cavity mirror comprising multiple reflection points, the multiple reflection points of the first cavity mirror or the second cavity mirror comprising at least one output reflection point;
wherein a light beam is transmitted into the optical resonant cavity through the input reflection point, and after the light beam is reflected N times between the multiple reflection points of the first cavity mirror and the multiple reflection points of the second cavity mirror, a re-incident condition is satisfied and then a next reflection cycle is entered, and such cycle is repeated until an energy of the light beam in the optical resonant cavity is attenuated to 0, wherein N≥4, and the re-incident condition is that a reflection position and a reflection angle of the light beam in the optical resonant cavity are the same as a transmission position and a transmission angle of the light beam when the light beam is first transmitted into the optical resonant cavity; and
at least one of the at least one input reflection point and the at least one output reflection point is a target reflection point, a transmittance of the target reflection point is greater than or equal to T, and transmittances of remaining reflection points are equal to T₀, wherein T>T₀>0.

2. The optical resonant cavity according to claim 1, wherein at least one of the at least one input reflection point has a transmittance being greater than or equal to T, and the second cavity mirror comprises N/2 output reflection points.

3. The optical resonant cavity according to claim 2, wherein T=mT₀, m=(N−1)/2, and m>1.

4. The optical resonant cavity according to claim 1, wherein one of the at least one input reflection point and one of the at least one output reflection point respectively have a transmittance being greater than or equal to T.

5. The optical resonant cavity according to claim 4, wherein T=mT₀, m=N−2, and m>1.

6. The optical resonant cavity according to claim 1, wherein one of the at least one input reflection point or one of the at least one output reflection point has a transmittance being greater than or equal to T.

7. The optical resonant cavity according to claim 6, wherein T=mT₀, m=(N−1)/2, and m>1.

8. The optical resonant cavity according to claim 1, wherein at least one of the at least one input reflection point has a transmittance being greater than or equal to Tₘ, and at least one of the at least one output reflection point has a transmittance being greater than or equal to Tₒᵤₜ, wherein Tᵢₙ≠Tₒᵤₜ, Tᵢₙ≥T, and Tₒᵤₜ≥T.

9. The optical resonant cavity according to claim 1, wherein for a target cavity mirror from the first cavity mirror and the second cavity mirror, reflection points with different transmittances are formed on the target cavity mirror based on an integrated coating method or a split coating method, and the target cavity mirror comprises multiple reflection points with different transmittances.

10. The optical resonant cavity according to claim 9, wherein based on the integrated coating method, a method for forming the multiple reflection points with different transmittances on the target cavity mirror is that: different film layers in different areas of the target cavity mirror are generated by using a mask in an integrated coating process; and
based on the split coating method, a method for forming the multiple reflection points with different transmittances on the target cavity mirror is that: different areas of the target cavity mirror are separated into independent components and the different components are coated separately in a split coating process.

11. The optical resonant cavity according to claim 1, wherein the optical resonant cavity further comprises:
at least one folding reflector, a reflection surface of the at least one folding reflector is arranged opposite to the reflection surface of the first cavity mirror or the reflection surface of the second cavity mirror, and the at least one folding reflector comprises multiple reflection points; and
after the light beam is transmitted into the optical resonant cavity through the input reflection point, and is reflected M times between the multiple reflection points of the first cavity mirror, the multiple reflection points of the at least one folding reflector and the multiple reflection points of the second cavity mirror, the re-incident condition is satisfied and the next reflection cycle is entered, and such cycle is repeated until the energy of the light beam is attenuated to 0, wherein M>N.

12. The optical resonant cavity according to claim 11, wherein at least one of all the multiple reflection points of the first cavity mirror or the second cavity mirror is an output reflection point, and the output reflection point is the target reflection point.

13. The optical resonant cavity according to claim 1, wherein at least one of the first cavity mirror and the second cavity mirror is a concave reflector.

14. A gas absorption spectrum detection device, comprising:
an optical resonant cavity, comprising:
a first cavity mirror, the first cavity mirror comprising multiple reflection points, the multiple reflection points of the first cavity mirror comprising at least one input reflection point; and
a second cavity mirror, a reflection surface of the second cavity mirror arranged opposite to a reflection surface of the first cavity mirror, the second cavity mirror comprising multiple reflection points, the multiple reflection points of the first cavity mirror or the second cavity mirror comprising at least one output reflection point;
wherein a light beam is transmitted into the optical resonant cavity through the input reflection point, and after the light beam is reflected N times between the multiple reflection points of the first cavity mirror and the multiple reflection points of the second cavity mirror, a re-incident condition is satisfied and then a next reflection cycle is entered, and such cycle is repeated until an energy of the light beam in the optical resonant cavity is attenuated to 0, wherein N≥4, and the re-incident condition is that a reflection position and a reflection angle of the light beam in the optical resonant cavity are the same as a transmission position and a transmission angle of the light beam when the light beam is first transmitted into the optical resonant cavity; and
at least one of the at least one input reflection point and the at least one output reflection point is a target reflection point, a transmittance of the target reflection point is greater than or equal to T, and transmittances of remaining reflection points are equal to T₀, wherein T>T₀>0; and
a photodetector, wherein the photodetector is configured to measure a light intensity of the light beam transmitted through the output reflection point, to obtain absorption spectrum information of a gas in the optical resonant cavity according to the light intensity or a ring-down time of the light intensity.

15. The gas absorption spectrum detection device according to claim 14, further comprising a converging lens, wherein the light beam, after being transmitted to the converging lens through the output reflection point, is converged to the photodetector through the converging lens.

16. The gas absorption spectrum detection device according to claim 14, further comprising a converging lens and a receiving optical fiber, wherein the light beam, after being transmitted to the converging lens through the output reflection point, is converged to the receiving optical fiber through the converging lens and transmitted to the photodetector.

17. The gas absorption spectrum detection device according to claim 14 wherein the gas absorption spectrum detection device is implemented based on a cavity ring-down spectroscopy technology, an incoherent broadband cavity enhanced absorption spectroscopy technology or an off-axis integral cavity output spectroscopy technology.

18. The gas absorption spectrum detection device according to claim 14, wherein at least one of the at least one input reflection point has a transmittance being greater than or equal to T, and the second cavity mirror comprises N/2 output reflection points, wherein T=mT₀, m=(N−1)/2, and m>1.

19. The gas absorption spectrum detection device according to claim 14, wherein one of the at least one input reflection point and one of the at least one output reflection point respectively have a transmittance being greater than or equal to T, wherein T=mT₀, m=N−2, and m>1.

20. The gas absorption spectrum detection device according to claim 14, wherein one of the at least one input reflection point or one of the at least one output reflection point has a transmittance being greater than or equal to T, wherein T=mT₀, m=(N−1)/2, and m>1.
